(19) United States
(12) Patent Application Publication
(10) Pub. No.: US 2010/0303754 A1
(43) Pub. Date: Dec. 2, 2010

(54) PROCESS FOR PREPARATION OF CYCLODEXTRIN OLIGOMERS OR POLYMERS, PRODUCTS OBTAINED AND USES

(75) Inventors: Frederic Turpin, Tours (FR); Carole Brigand, Poitiers (FR); Yves Cenatiempo, Saint Julien L'ars (FR); EI Mustapha Belgsir, Poitiers (FR)

Correspondence Address: YOUNG & THOMPSON, 209 Madison Street, Suite 500, Alexandria, VA, 22314, US

(73) Assignee: BIOCYDEX, POITIERS (FR)

(21) Appl. No.: 12/446,309

(22) PCT Filed: Oct. 18, 2007
(86) PCT No.: PCT/FR07/52195

§ 371 Date: Apr. 20, 2009

(30) Foreign Application Priority Data
Oct. 20, 2006 (FR) 06 54412

(51) Int. Cl. A61K 38/20; C07H 3/00; C07K 2/00; C07K 14/00; C07H 21/00; C07H 3/06; A61K 31/52; A61K 31/505; A61K 31/515; A61K 8/73; A61K 38/00; A61K 31/56; A61K 38/21; A61P 31/00; A61P 9/00; A61P 25/00; A61P 33/00; A61P 35/00

(52) U.S. Cl. 424/85.2; 536/103; 530/322; 530/350; 536/23.1; 536/123.1; 514/263.1; 514/256; 514/270; 514/9.7; 514/1.1; 514/169; 424/85.4

(57) ABSTRACT

A process for the preparation of cyclodextrin oligomers or polymers, whereby the cyclodextrin molecules are coupled to one another covalently via a spacer arm, based on a coupling reaction between an alkyne and an azide producing the formation of an aromatic heterocyclic bridge between the coupled units. Also described are the cyclodextrin oligomers or polymers that are obtained and their uses.

This invention relates to a process for preparation of cyclodextrin oligomers or polymers.

The invention also relates to the cyclodextrin oligomers or polymers that are obtained and their uses.

The cyclodextrins, or cyclomaltooligosaccharides, are cyclic oligosaccharides that have a tapered structure, with a hydrophilic outer surface and a relatively hydrophobic inner surface.

Because of this particular structure, the cyclodextrins have numerous properties, the most noteworthy being their capability of including various, preferably hydrophobic-type, molecular structures in their cavity to form water-soluble inclusion complexes.

This specificity has given rise to applications in numerous fields, in particular in pharmacy, in particular for the transport of medications, in agrochemistry, in cosmetics, in the industry of perfume and scents, or else in the environment.

For the purpose of improving the complexing properties of cyclodextrins, it is possible to resort to the synthesis of cyclodextrin oligomers or polymers. These structures consist of several cyclodextrin molecules that are coupled or cross-linked together covalently to allow cooperative complexing with one or more invited molecule(s).

A cyclodextrin oligomer is defined as a molecule that consists of a small finite number of cyclodextrin monomers that are coupled or cross-linked together covalently.

A cyclodextrin polymer is defined as a molecule that consists of infinite cyclodextrin monomers that are coupled or cross-linked together covalently.

The cyclodextrin oligomers or polymers are structures of choice for numerous applications, in particular for the solubilization of substances that are pharmacologically active in aqueous media that are difficult to complex with simple cyclodextrins.

Several examples have already been described.

It is possible to cite the application EP-1,689,789 that mentions processes for preparation of cyclodextrin dimers that are able to complex and to solubilize the anti-cancer agents of the taxoid family. These compounds can contain glucidic substituents that impart a particular affinity to the dimer for certain biological sites.

The U.S. Pat. No. 6,660,804 that describes a process for preparation of cyclodextrin copolymers based on derivatives of cyclodextrins and polycarboxylic acids is also known.

The application EP-1,183,538 relative to the preparation of a controlled-release pharmaceutical composition based on cleavable cyclodextrin polymers that can be coupled to biorecognition molecules is also known.

The processes for synthesis of known cyclodextrin oligomers or polymers rely on common coupling reactions.

These processes are complex and require the identification of functional groups that are suited to each cyclodextrin and the implementation of suitable chemical methods.

They produce primarily the formation of amide groups (—NH—CO—), ester groups (—O—CO—), urea groups (—NH—CO—NH—), thiourea groups (—NH—CS—NH—), imide groups (═N—; —N═), amine groups (—NH—), ether groups (—O—), thioether and disulfide groups (—S—S—), and groups that are sensitive to hydrolysis, to oxidation, or to certain chemical and enzymatic metabolizations.

These synthesis processes generally use toxic and volatile organic solvents and comprise long and complicated purification stages.

Their implementation is therefore burdensome and very expensive.

In addition, these processes are not very effective and make possible only low yields.

The processes for preparation of existing cyclodextrin oligomers or polymers therefore have major drawbacks that make them difficult to use on the industrial scale.

A need therefore persists for a process for preparation of cyclodextrin oligomers or polymers that is simple, effective, ecological, and inexpensive at the same time.

This is the purpose of this invention in proposing a process for preparation of cyclodextrin oligomers or polymers, whereby the cyclodextrin molecules are coupled to one another covalently via a spacer arm, based on a coupling reaction between an alkyne and an azide that bring about the formation of an aromatic heterocyclic bridge between the coupled units. More particularly, this invention proposes a process that is flexible and simple to implement, which uses the Huisgen 1,3-dipolar cycloaddition reaction.

The invention is now described in detail.

In this description, “cyclodextrin” is defined as any natural or modified cyclodextrin.

Likewise, “spacer arm” refers to any multi-branch multiplication element onto which azide or alkyne groups can be integrated.

According to one aspect of the invention, the cyclodextrins that are used for the preparation of the same cyclodextrin oligomer or polymer can be identical or different.

Preferably, the cyclodextrins that are used for the preparation of an oligomer or polymer according to the invention correspond to the following formula:

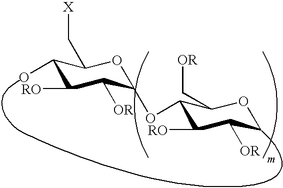

in which:
m represents a whole number that is equal to 5, 6 or 7, and
the groups R, identical or different, represent a hydrogen atom or an acyl, alkyl, hydroxyalkyl or sulfoalkyl group of 1 to 16 carbon atoms.

Preferably, the spacer arm(s) is/are selected from among the hydrocarbon groups, the peptides, the proteins, the oligonucleotides, the polynucleotides, the oligosaccharides, the polysaccharides, or the biopolymers. The hydrocarbon groups can be aliphatic or aromatic, saturated or unsaturated, optionally substituted with heteroatoms that are selected from among O, S or N.

Still more preferably, the spacer arm(s) are glycols, diethylene glycols, triethylene glycols or molecules of 1,1,1-tris(hydroxymethyl)ethane, 1,1,1-tris(hydroxymethyl)-aminomethane or pentaerythritol.

The alkyne groups that are used according to this invention can be mono- or di-substituted, symmetrical or asymmetrical.

### I/Process for Preparation of Cyclodextrin Oligomers or Polymers According to the Invention

The purpose of this invention is a process for preparation of cyclodextrin oligomers or polymers, whereby the cyclodextrin molecules are coupled to one another covalently via a spacer arm, comprising at least:
One stage for integration of azide and/or alkyne groups onto the cyclodextrins and/or onto the spacer arm(s), and
One stage for creating bonds between the spacer arm(s) and the cyclodextrins.
   1. Stage for Integration of Alkyne and Azide Groups onto Each Cyclodextrin and/or onto Each Spacer Arm

This stage consists in integrating:
At least one alkyne group onto each cyclodextrin so as to obtain acetylene cyclodextrins and at least two azide groups on each spacer arm, or
At least one azide group onto each cyclodextrin so as to obtain azide cyclodextrins and at least two alkyne groups on each spacer arm.

The azide and alkyne groups are easily integrated in cyclodextrins and in spacer arms. These are stable groups in a majority of organic synthesis conditions.

According to a particular embodiment of the invention, the azide or acetylene cyclodextrins that are formed can undergo other chemical transformations such as an alkylation, a halogenation, an esterification, etc., without resorting to any additional stage of protection and/or deprotection.

Thus, the process for preparation of cyclodextrin oligomers or polymers according to the invention can also comprise a stage for chemical transformation of azide or acetylene cyclodextrins before the stage for creating bonds between the spacer arms and the cyclodextrins.

### 2. Stage for Creating Bonds Between the Spacer Arm(s) and the Cyclodextrins

Once the alkyne and azide groups are attached to the cyclodextrins and the spacer arm(s), the process according to the invention provides a stage for creating bonds between the spacer arm(s) and the cyclodextrins in the form of 1,2,3-triazole cycles by coupling reaction between the alkynes and the azides.

The reaction that is produced, also called a Huisgen 1,3-dipolar cycloaddition reaction, is as follows:

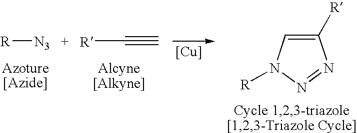

This reaction makes it possible to carry out simple and reliable chemical transformations. It is an effective and highly versatile reaction that makes possible the construction of a large variety of multivalent structures.

This coupling reaction can be carried out in an aqueous medium, with relatively short reaction times and high yields.

Thus, advantageously, the process for preparation of cyclodextrin oligomers or polymers according to the invention is simple to use.

This process can be produced in an aqueous medium under mild reaction conditions.

This process is effective, ecological, and inexpensive, and the yields that are obtained are high.

The process according to the invention can be used for any cyclodextrin, without having to find suitable functional groups and having to implement chemical methods that are suited to each type of cyclodextrin.

Another advantage of this invention is its synthetic flexibility. Actually, the preparation process according to the invention makes it possible to consider the synthesis of cyclodextrin oligomers or polymers with variable spacer arm lengths so as to adapt the geometry of the cyclodextrin oligomer or polymer for an effective and even optimal complexing of one or more invited molecule(s) that is/are determined.

II/Cyclodextrin Oligomers or Polymers that are Obtained According to the Invention

The cyclodextrin oligomers or polymers that can be obtained by the implementation of the process according to the invention correspond to the following general formula:

CD-X-A-T-A-X—B(—X-A-T-A-X-CD)ₙ

in which:
CD, identical or different, represent a cyclodextrin, linked by its secondary or primary face,
X, identical or different, represent an amine group (—NH—), an ether group (—O—), a thioether group (—S—), a disulfide group (—S—S—), a carbamate group (—NHCO—O—; —O—CO—NH—), a carboxylic or sulfonic ester group (—O—CO—; —O—SO₂), a carboxylic or sulfonic amide group (—NHCO—; —NHSO₂—), an imide group (—N═; ═N—) or a group T,
A, identical or different, represent a linear or branched C1 to C10 aliphatic group, a substituted or non-substituted mono- or polycyclic aromatic group, or a group T,
T represents a 1,2,3-triazole cycle,
B represents:
   A multi-branch multiplication element, whereby the branches comprise at least one biological recognition group, or at least
   A probe for fluorescent or radioactive visualization or detection,
   A linear or branched aliphatic group,
   A mono- or polycyclic aromatic group that is substituted or non-substituted,
   An atom of carbon, oxygen, sulfur, nitrogen or phosphorus,
   A polymer,
   A solid substrate (silica, resin, . . . ), or
   A group CD, X, A and/or T, and
n represents a whole number that is greater than or equal to 1.

When X=A, the associated group X-A-T or T-A-X represents a group T.

“Multi-branch multiplication element” is defined as a functionalized symmetrical or asymmetrical compound that comprises at a minimum two branches, such as, for example, an ethylene glycol chain, a polyethylene glycol chain or a molecule of TRIS (tris(hydroxymethyl)aminomethane or tris(hydroxymethyl)ethane) or pentaerythritol.

A “group of biological recognition” is an additional molecular structure of a biological receptor, able to be recognized by the latter and to lead to a specific response, such as, for example, the induction and the regulation of the biosynthesis of an enzyme, the inhibition of the enzymatic activity of an enzyme by attachment to its active site, the induction of an immune response following a bacterial infection, or else the inhibition of an inflammatory process by blockage of the active site.

The expression “probe for fluorescent or radioactive visualization or detection” refers to any molecular structure that makes possible the detection of a system by a physicochemical technique, such as fluorescence or radioactivity.

The cyclodextrin oligomers or polymers that are obtained according to the invention can be symmetrical or non-symmetrical. For the symmetrical compounds, the groups CD, A, X and T are respectively identical on both sides of group B. For the asymmetrical compounds, one or more of the groups CD, A, X or T are different on both sides of the group B.

In the same oligomer or polymer, the cyclodextrins can be identical or different. Preferably, the cyclodextrins CD correspond to the following formula:

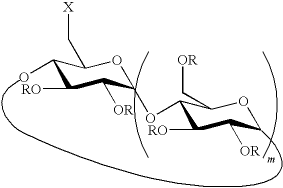

in which:
m represents a whole number that is equal to 5, 6 or 7, and
The groups R, identical or different, represent a hydrogen atom or an acyl group, an alkyl group, a hydroxyalkyl group, or a sulfoalkyl group with 1 to 16 carbon atoms.

Advantageously, the oligomers or polymers according to the invention comprise triazole cycles that contribute to improving the complexing of the oligomers or polymers with invited molecules. In addition, the triazole cycles resist hydrolysis and oxidation and are able to associate easily with biological targets by dipolar interactions and hydrogen bonds.

### III/Uses

The cyclodextrin oligomers or polymers can be used for a cooperative complexing with one or more invited molecules.

In particular, the cyclodextrin oligomers or polymers according to the invention can be used to solubilize and vectorize in aqueous medium one or more hydrophobic chemical compound(s), in particular one or more pharmacologically and/or cosmetologically active substance(s).

Even more particularly, the cyclodextrin oligomers or polymers according to the invention can be used for the effective and selective transport of one or more pharmacologically active substance(s) to one or more target organ(s).

The pharmacologically active substance(s) can be selected from among the anti-cancer medications, the anti-tumor medications, the anti-fungal medications, the antibacterial medications, the anti-viral medications, the cardiovascular medications, the neurological medications, the alkaloids, the antibiotics, the bioactive peptides, the steroids, the steroid hormones, the polypeptide hormones, the interferons, the interleukins, the narcotics, the prostaglandins, the purines, the pyrimidines, the anti-protozoan medications, the barbiturates, or the anti-parasitic medications.

According to another aspect, the cyclodextrin oligomers or polymers obtained according to the invention can also be used for the design of materials based on cyclodextrins that are immobilized on porous substrates, such as silicas or resins, for example. They can also be used for the attachment and the separation of various substances, in particular by chromatographic techniques, for example.

### IV/Examples of Preparation of Cyclodextrin Dimers According to the Invention

The examples of cyclodextrin dimers that will follow are obtained by reacting two monoazide cyclodextrins with a spacer arm that is functionalized at its ends by alkynes.

The general diagram of formation of cyclodextrin dimers according to the invention is as follows:

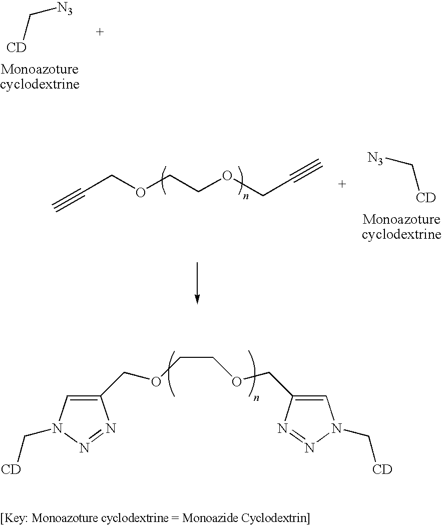

The examples of cyclodextrin dimers according to the invention are obtained from monoazide-β-cyclodextrins for the examples DM1, DM2, and DM3, and the alkylated monoazide-β-cyclodextrins for DM4.

The monoazide-β-cyclodextrin is synthesized as follows:
Selective monotosylation of the β-cyclodextrin in the presence of tosyl chloride (TsCl) in an alkaline aqueous solution, and
Conversion of the tosyl group into azide by nucleophilic substitution with an azidation agent, such as, for example, sodium azide, lithium azide, barium azide, azidotrimethylsilane or sulfonyl azides.

The alkylated monoazide-β-cyclodextrin is synthesized as follows:
Selective monotosylation of the β-cyclodextrin in the presence of tosyl chloride (TsCl) in an alkaline aqueous solution,
Conversion of the tosyl group into azide by nucleophilic substitution with an azidation agent, such as, for example, sodium azide, lithium azide, barium azide, azidotrimethylsilane or sulfonyl azides, and
Selective methylation of the hydroxyl groups that are located in positions 2 and 6.

The examples of cyclodextrin dimers according to the invention are obtained with spacer arms of different lengths for the examples DM1, DM2 and DM3 and of identical lengths for the examples DM1, DM4 and DM5.

The spacer arms are formed from glycol and polyethylene glycol chains, selected for their chemical stability and their biological compatibility. They are functionalized at their ends by alkynes.

The spacer arms are synthesized in the presence of propargyl bromide and sodium hydride at ambient temperature in tetrahydrofuran (THF).

The 1,3-dipolar cycloaddition is carried out in the presence of a pair of catalysts that makes possible the exclusive formation of the 1,4-triazole product.

The commonly used catalysts are copper sulfate or copper bromide, combined with bases such as sodium L-ascorbate (L-asc.) or diisopropylethylamine (DIPEA).

Preferably, the copper sulfate/L-asc. pair is used in an aqueous medium. The CLHP (high-performance liquid chromatography) analyses of the crude dimers show that the yields are quasi-quantitative.

The dimers that are obtained can be used without additional purifications.

### 1. Example of Synthesis of a Monoazide β-Cyclodextrin: 6^{I}-Azido-6^{I}-deoxy-cyclomatoheptaose

In a flask that is provided with a coolant and a nitrogen intake, the monotosyl β-cyclodextrin is solubilized slowly in DMF (dimethylformamide), dried in advance in a molecular sieve. Sodium azide is added, and the batch is stirred at 60° C. under inert atmosphere.

At the end of the reaction, the DMF is evaporated, and the residue is taken up in ethanol: a white precipitate appears.

The white precipitate is filtered on sintered glass and washed with ethanol. A white powder that is dried under forced vacuum is obtained.

The monoazide-β-cyclodextrin that is obtained can be used without additional purification for the production of DM1, DM2 or DM3.

### 2. Examples of Synthesis of Alkylated Monoazide β-Cyclodextrins

### 2.1 Synthesis of an Alkylated Monoazide β-Cyclodextrin: 6^{I}-Azido-6^{I}-deoxy-2^{I}-O-methyl-hexakis[2^{II-VII},6^{I-VII}di-O-methyl]cyclomaltoheptaose

In a reactor that is provided with a coolant, a nitrogen intake, and an addition ampoule, the monoazide β-cyclodextrin that is obtained in 1 is solubilized in dry DMF.

Barium hydroxide and barium oxide are added to the reaction mixture.

The batch is cooled to 0° C., and dimethyl sulfate is added drop by drop.

The reaction is left for 18 hours at 0° C.

When the reaction is finished, 25% aqueous ammonia is added.

The reaction mixture is heated to 70° C. for two hours, and then the solvents are evaporated.

The residue is taken up with dichloromethane, the precipitate that is formed is filtered on sintered glass with a porosity of 3, and it is washed with dichloromethane.

The filtrate is recovered and then extracted with water and with brine.

The organic phase that is collected is dried on anhydrous sodium sulfate.

After filtration and then evaporation, the crude product is dried under forced vacuum. The residue is covered by methanol and then placed at 6° C.

The white precipitate that is obtained is filtered, washed with methanol, and then dried under forced vacuum.

The alkylated monoazide-β-cyclodextrin that is obtained can be used without additional purification for the production of the dimer DM4 and the trimer TM 1.

### 2.2 Synthesis of an Alkylated Monoazide β-Cyclodextrin: 6^{I}-Azido-6^{I}-deoxy-3^{I}-O-methyl-hexakis[2^{II-VII},3^{II-VII}-di-O-methyl]cyclomaltoheptaose

In a reactor that is provided with a coolant, a nitrogen intake, and an addition ampoule, the monoazide β-cyclodextrin that is obtained in 1 is solubilized in anhydrous DMF.

Sodium hydride is added to the reaction mixture.

The batch is cooled to 0° C., and methyl iodide is slowly added.

The reaction is kept at ambient temperature for 24 hours.

When the reaction is finished, the reaction medium is filtered, and the filtrate is concentrated under vacuum. The oily residue is taken up in a minimum amount of water and then extracted with dichloromethane.

The organic phase that is collected is washed with water, with brine, and it is dried on anhydrous sodium sulfate.

After evaporation of the solvent under vacuum, the oily residue is taken up in a minimum amount of water, the insoluble products are filtered, and the solution is freeze-dried.

After filtration, then evaporation, the crude product is dried under forced vacuum. The residue is covered by methanol and then placed at 6° C.

The product is obtained in the form of a white powder.

The alkylated monoazide-β-cyclodextrin that is obtained can be used without additional purification for the production of the dimer DM5.

### 3. Examples of Syntheses of Spacer Arms

### 3.1 Synthesis of Dialkyne Spacer Arms from Glycol, Diethylene or Triethylene Glycol

In a flask, the glycol chain (glycol, diethylene glycol or triethylene glycol) is solubilized in THF (tetrahydrofuran or 1,4-epoxybutane).

Sodium hydride is added, and the mixture is stirred for several minutes under inert atmosphere.

The reaction mixture is cooled to 0° C. in an ice water bath, and propargyl bromide is added drop by drop.

At the end of the reaction, distilled water is added to the reaction medium so as to hydrolyze the excess propargyl bromide.

The solvents are evaporated, and the oil that is obtained is taken up in ethyl acetate and then washed with water.

The organic phase is recovered, dried with sodium sulfate, and then filtered.

Ethyl acetate is evaporated, and the residue that is obtained is purified on a silica gel column with a pentane/ether mixture as an eluant phase.

The product that is obtained comes in the form of a yellow oil.

### 3.2 Synthesis of Trialkyne Spacer Arms from 1,1,1-Tris(Hydroxymethyl)Ethane

In a flask, 1,1,1-tris(hydroxymethyl)ethane is solubilized in THF.

Sodium hydride is added, and the mixture is stirred for several minutes under inert atmosphere.

The reaction mixture is cooled to 0° C. in an ice water bath, and propargyl bromide is added drop by drop.

At the end of the reaction, distilled water is added to the reaction medium so as to hydrolyze the excess propargyl bromide.

The solvents are evaporated, and the oil that is obtained is taken up in ethyl acetate and then washed with water.

The organic phase is recovered, dried with sodium sulfate, and then filtered.

Ethyl acetate is evaporated, and the residue that is obtained is purified on a silica gel column with a pentane/ether mixture as an eluant phase.

The product that is obtained comes in the form of a yellow oil.

### 3.3 Synthesis of the Branched Dialkyne Spacer Arm from Tert-Butyl [3-(Di-Prop-2-ynyl-amino)-propyl]-carbamate

In a flask that is topped with an isobaric flow ampoule, N-(tert-butoxycarbonyl)-1,3-diaminopropane is solubilized in THF.

Triethylamine is then added.

The flask is immersed in an ice bath, and propargyl bromide that is solubilized in THF is added drop by drop.

After 24 hours of reaction, the salts that are formed are filtered, and the filtrate is taken up in water and then stirred for one hour to hydrolyze the excess propargyl bromide.

The water and the THF are evaporated under vacuum, and the residue that is obtained is taken up in dichloromethane. The solution is washed with water with a dilute solution of soda and brine.

The organic phase is recovered, dried on anhydrous sodium sulfate, and then filtered. The product is purified on a silica gel column.

The reaction diagram of this synthesis is as follows:

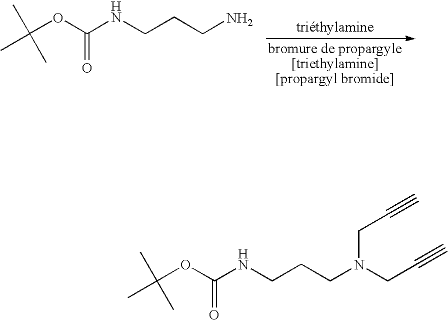

### 4. Synthesis of Oligomers

### 4.1 Synthesis of Dimers DM1, DM2, DM3, DM4, DM5 and Trimer TM1

As azide cyclodextrin precursors:
Monoazide-β-cyclodextrins, as synthesized in 1, for DM1, DM2, and DM3,
Alkylated monoazide-β-cyclodextrins as synthesized in 2.1, for DM4 and TM1, and
Alkylkated monoazide-β-cyclodextrins as synthesized in 2.2, for DM5, are used.

As spacer arms:
Dialkyne glycol as synthesized in 3.1 for DM1, DM4 and DM5,
Dialkyne diethylene glycol as synthesized in 3.1 for DM2,
Dialkyne triethylene glycol as synthesized in 3.1 for DM3, and
Trialkyne TRIS as synthesized in 3.2 for TM1 are used.

In a flask that is topped with a coolant, the azide cyclodextrin precursors are solubilized in distilled water.

L-Asc. and pentahydrated copper sulfate are then added.

The glycol derivative, solubilized in ethanol, is added to the reaction mixture, and the batch is heated to 70° C.

After several hours of stifling, the reaction medium becomes perfectly clear green. The solvents are evaporated, and the residue that is obtained is taken up in ethanol.

A precipitate that is filtered and then rinsed is formed.

The colored powder that is obtained is solubilized in distilled water and then brought into the presence of Amberlite 200.

After filtration, the solution is freeze-dried: the dimers DM1, DM2, DM3, DM4, DM5 and the trimer TM1 are obtained in the form of a slightly colored powder, which does not require additional purification.

### 4.2 Synthesis of the Dimer DM6

In a first step, a branched monoazide cyclodextrin is synthesized from alkylated monoazide-β-cyclodextrins as synthesized in 2.1 and from tert-butyl [3-di-prop-2-ynyl-amino)-propyl]-carbamate as synthesized in 3.3.

The reaction diagram is as follows:

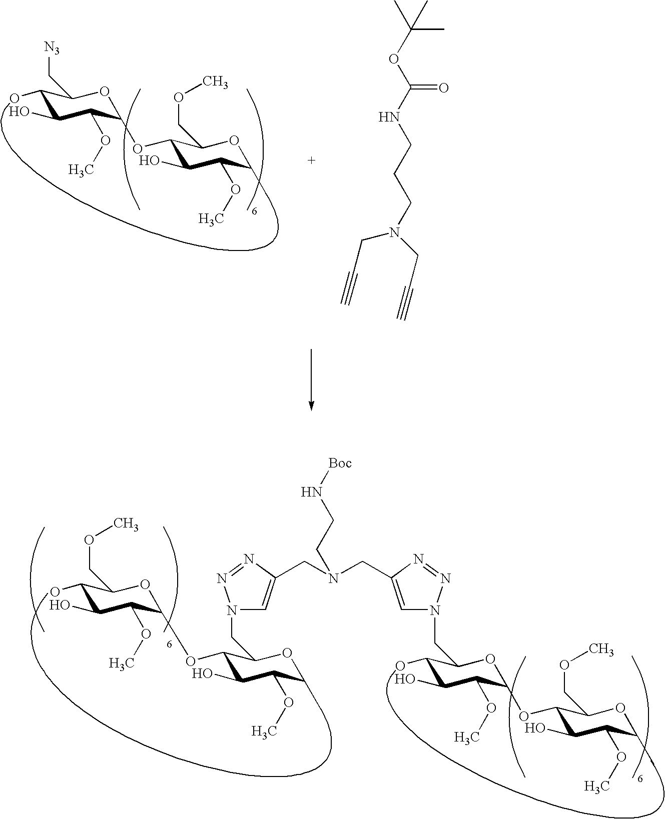

An alkylated monoazide-β-cyclodextrin as synthesized in 2.1 is solubilized in a water-ethanol mixture with L-ascorbate, copper sulfate, and tert-butyl [3-(di-prop-2-ynyl-amino)-propyl]-carbamate as synthesized in 3.3.

The reaction medium is heated to 50° C. When the reaction is finished, the ethanol is evaporated. The aqueous solution is extracted with dichloromethane. The organic phase is then washed with a dilute hydrochloric acid solution, and then brine. The organic phase is recovered, dried on anhydrous sodium sulfate, filtered and evaporated under vacuum. The residue that is obtained is purified on a silica gel column and then used to synthesize the dimer DM6.

DM6 is obtained by coupling a folic acid on the branched monoazide cyclodextrin obtained above. The reaction diagram is as follows:

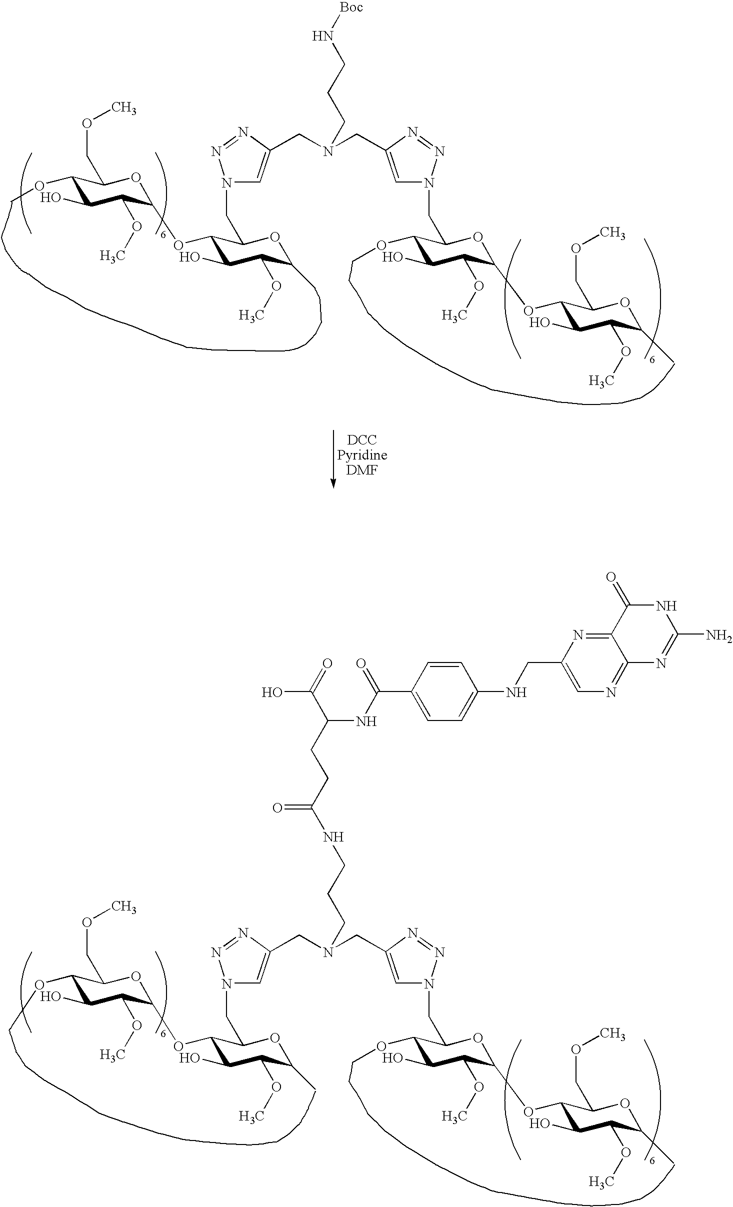

The branched monoazide cyclodextrin is solubilized in dichloromethane, and then TFA (trifluoroacetic acid) is added.

The reaction is stirred for 20 hours.

The solution is washed with water, with a solution of dilute soda, and then with a brine solution.

The organic phase is dried and then evaporated under vacuum, and the product is dried under forced vacuum. The residue that is obtained is placed in a flask that is protected from light and then is solubilized in DMF. DCC(N,N′-dicyclohexyl-carbodiimide) folic acid and a catalytic amount of pyridine are added to this solution. When the reaction is finished, the DMF is evaporated under vacuum, and the residue is taken up in dichloromethane. The solution is washed with water, with a solution of dilute soda and with brine. The organic phase is dried on anhydrous sodium sulfate, filtered and evaporated. The product is purified on a silica gel column.

### 5. Solubilization of Molecules by DM1, DM2, DM3, DM4, DM6 and TM1

Solubilization tests are carried out by bringing solutions containing 50 mmol of DM1, DM2, DM3, DM4, DM6 and TM1 into the presence of known molecules that can be used as active ingredients in initial molar ratios of 1 active ingredient per 10 cyclodextrin dimers.

Each solution is stirred for 24 hours, and then the soluble fractions are analyzed by CLHP after ultracentrifuging and filtration.

The amounts of active ingredients that are solubilized in g/L are presented in the following table:

| | | | | Mometasone | | | | Fluticasone |
|---|---|---|---|---|---|---|---|---|
| | Taxol | Taxotere | Cyclosporin A | Furoate | Astaxanthin | Voriconazole | Roxithromycin | Propionate |
| Water | 0.0007 | 0.004 | 0.022 | 0 | 0 | 0.6854 | 0.4645 | 0.0001 |
| DM1 | 0.0025 | 1.44 | 0.404 | 0.032 | 0.011 | 6.960 | 14.693 | 0.0638 |
| DM2 | 0.0029 | 1.63 | 0.393 | 0.034 | 0.001 | 0.680 | 14.640 | 0.0654 |
| DM3 | 0.008 | 0.34 | ND | ND | ND | ND | ND | ND |
| DM4 | 1.64 | 10.8 | 4.473 | 5.554 | 0.806 | ND | ND | 5.097 |
| DM6 | ND | 10.1 | ND | ND | ND | ND | ND | ND |
| TM1 | ND | 23.1 | ND | ND | ND | ND | ND | ND |

With ND: Not Determined

It is noted that the dimers that are prepared according to the invention make it possible to increase the solubility of the tested active ingredients significantly.

Of course, the invention obviously is not limited to the examples that are shown and described above but on the contrary covers all of the variants, in particular with regard to the nature of cyclodextrins and spacer arms, as well as the uses of the cyclodextrin oligomers or polymers that are obtained.

1. Process for preparation of cyclodextrin oligomers or polymers, whereby the cyclodextrin molecules are coupled to one another covalently via a spacer arm, characterized in that it comprises at least the following stages:
Integration of at least one alkyne group on each cyclodextrin so as to obtain acetylene cyclodextrins and at least two azide groups on each spacer arm, and
Creation of bonds between the spacer arm(s) and the cyclodextrins in the form of 1,2,3-triazole cycles by coupling reaction between the alkynes and the azides.

2. Process for preparation of cyclodextrin oligomers or polymers, whereby the cyclodextrin molecules are coupled to one another covalently via a spacer arm, wherein it comprises at least the following stages:
Integration of at least one azide group on each cyclodextrin so as to obtain azide cyclodextrins and at least two alkyne groups on each spacer arm, and
Creation of bonds between the spacer arm(s) and the cyclodextrins in the form of 1,2,3-triazole cycles by coupling reaction between the alkynes and the azides.

3. Process for preparation of cyclodextrin oligomers or polymers according to claim 1, wherein it comprises at least one stage for chemical transformation of azide cyclodextrins or acetylene cyclodextrins before the stage of creating bonds between the spacer arms and the cyclodextrins.

4. Process for preparation of cyclodextrin oligomers or polymers according to claim 1, wherein the spacer arm(s) is/are selected from among the hydrocarbon groups, the peptides, the proteins, the oligonucleotides, the polynucleotides, the oligosaccharides, the polysaccharides, or the biopolymers.

5. Process for preparation of cyclodextrin oligomers or polymers according to claim 1, wherein the spacer arm(s) is/are selected from among glycol, diethylene glycol, or triethylene glycol.

6. Process for preparation of cyclodextrin oligomers or polymers according to claim 1, wherein the cyclodextrins correspond to the following formula: 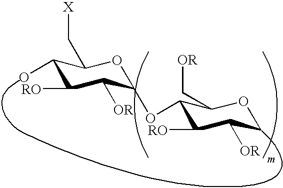
in which:
m represents a whole number that is equal to 5, 6 or 7, and
The groups R, identical or different, represent a hydrogen atom or an acyl, alkyl, hydroxyalkyl or sulfoalkyl group of 1 to 16 carbon atoms.

7. Process for preparation of cyclodextrin oligomers according to claim 1, wherein the stage for integrating alkyne and azide groups into the cyclodextrins and the spacer arms consists in synthesizing monoazide-β-cyclodextrins or alkylated monoazide-β-cyclodextrins and in synthesizing dialkyne glycols, dialkyne diethylene glycols, dialkyne triethylene glycols, trialkyne glycols, or 1,1,1-tris(hydroxymethyl)ethane molecules, 1,1,1-tris(hydroxymethyl)aminomethane molecules or pentaerythritol molecules.

8. Cyclodextrin oligomer or polymer that is obtained by the implementation of the process according to claim 1, wherein it corresponds to the following general formula:
CD-X-A-T-A-X—B(—X-A-T-A-X-CD)ₙ
in which:
CD represents a cyclodextrin, linked by its secondary or primary face,
X represents an amine group, an ether group, a thioether group, a disulfide group, a carbamate group, a carboxylic or sulfonic ester group, a carboxylic or sulfonic amide group, an imide group, or a covalent bond,
A represents a linear or branched C1 to C10 aliphatic group, a substituted or non-substituted mono- or polycyclic aromatic group, or a covalent bond,
T represents a 1,2,3-triazole cycle,
B represents:
A multi-branch multiplication element, whereby the branches comprise at least one biological recognition group, or at least one probe for fluorescent or radioactive visualization or detection,
A linear or branched aliphatic group,
A mono- or polycyclic aromatic group that is substituted or non-substituted,
An atom of carbon, oxygen, sulfur, nitrogen or phosphorus,
A polymer,
A solid substrate (silica, resin, . . . ), or
A group C D, X, A and/or T, and
n represents a whole number that is greater than or equal to 1.

9. A cyclodextrin oligomer or polymer that is obtained by the implementation of the process according to claim 1 for a cooperative complexing with one or more invited molecules.

10. A composition comprising at least one hydrophobic chemical compound; an aqueous medium; and a cyclodextrin oligomer or polymer that is obtained by the implementation of the process according to claim 1 for solubilizing and vectorizing said at least one hydrophobic chemical compound in said aqueous medium.

11. The composition according to claim 10, wherein the at least one hydrophobic chemical compound is a pharmacologically and/or cosmetologically active substance.

12. A composition comprising at least one pharmacologically active substance; and a cyclodextrin oligomer or polymer that is obtained by the implementation of the process according to claim 1 for the selective transport of said at least one pharmacologically active substance to at least one target organ.

13. The composition according to claim 11, wherein the at least one pharmacologically active substance is selected from among the anti-cancer medications, the anti-tumor medications, the anti-fungal medications, the antibacterial medications, the anti-viral medications, the cardiovascular medications, the neurological medications, the alkaloids, the antibiotics, the bioactive peptides, the steroids, the steroid hormones, the polypeptide hormones, the interferons, the interleukins, the narcotics, the prostaglandins, the purines, the pyrimidines, the anti-protozoan medications, the barbiturates, or the anti-parasitic medications.

14. The composition according to claim 12, wherein the at least one pharmacologically active substance is selected from among the anti-cancer medications, the anti-tumor medications, the anti-fungal medications, the antibacterial medications, the anti-viral medications, the cardiovascular medications, the neurological medications, the alkaloids, the antibiotics, the bioactive peptides, the steroids, the steroid hormones, the polypeptide hormones, the interferons, the interleukins, the narcotics, the prostaglandins, the purines, the pyrimidines, the anti-protozoan medications, the barbiturates, or the anti-parasitic medications.
